(19) United States
(12) Patent Application Publication
(10) Pub. No.: US 2007/0082956 A1
(43) Pub. Date: Apr. 12, 2007

(54) The use of 1-amino-alkylcyclohexane compounds in the treatment of pain hypersensitivity.

(75) Inventors: Walter Magerl, Mainz (DE); Thomas Klein, Budenheim (DE); Rolf-Detlef Treede, Mainz (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: MERZ PHARMA GmbH & Co. KGaA, Frankfurt/Main (DE)

(21) Appl. No.: 10/566,106

(22) PCT Filed: Jul. 28, 2004
(86) PCT No.: PCT/IB04/03043

§ 371 Date: Jul. 10, 2006

Related U.S. Application Data
(60) Provisional application No. 60/490,865, filed on Jul. 28, 2003.
(60) Provisional application No. 60/526,825, filed on Dec. 2, 2003.
(60) Provisional application No. 60/537,215, filed on Jan. 16, 2004.

(51) Int. Cl. A61K 31/13

(52) U.S. Cl. 514/579; 514/659

(57) ABSTRACT

The invention relates to a novel use of 1-amino-alkylcyclohexane NMDA receptor antagonists such as neramexane in the treatment of pain hypersensitivity and neuropathic pain.

### FIELD OF THE INVENTION

The present invention relates to the use of 1-amino-alkylcyclohexane NMDA receptor antagonists in the treatment of pain hypersensitivity and neuropathic pain.

### BACKGROUND OF THE INVENTION

The nervous system routinely sends coded signals that result in sensation. Certain types of lesions to either the central or peripheral nervous system can result in an alteration of sensation resulting in pain. Pain is a sensation that hurts. It may cause discomfort or distress or agony. It may be steady or throbbing. It may be stabbing, aching, or pinching.

Pain is commonly defined as “an unpleasant sensation occurring in varying degrees of severity as a consequence of injury, disease, or emotional disorder.” Pain is a sensation that all people must deal with at some point. Although the statistics on pain are unknown, it is agreed upon that nearly all people experience pain at some point in their lives.

Pain has multiple causes. A familiar cause is trauma, such as a sprain or muscle injury or broken bone, or from surgery. Pain due to inflammation, such as a toothache, is also familiar to many. Headache is a common experience and arises often for unknown reasons. Cancer patients may have pain for a variety of reasons. It may be due to the effects of the cancer itself, or it could result from treatment methods.

Pain may be acute or chronic. Acute pain can be severe, but lasts a relatively short time. It is usually a signal that body tissue is being injured in some way, and the pain generally disappears when the injury heals. Chronic pain may range from mild to severe, and it is present to some degree for long periods of time. Chronic pain often arises without any detectable injury. Human persistent pain conditions can be classified into two categories: Complex Regional Pain Syndrome I (CRPS I) and Complex Regional Pain Syndrome II (CRPS II). CRPS I refers to pain without obvious nerve injury while CRPS II refers to pain with known nerve injury (Merskey, H. and N. Bogduk. 1994. Classification of Chronic Pain, Second Edition, IASP Press).

The difference between acute and chronic pain is discussed by Joseph T. Dipiro, Pharmacotherapy: A Pathophysiologic Approach, Third Edition, Appleton & Lange (1997) p. 1263. Dipiro explains that acute pain may be a useful physiologic process warning individuals of disease states and potentially harmful situations. Unfortunately, severe, unremitting, undertreated pain, when it outlives its biologic usefulness, can produce many deleterious effects such as psychological problems. When pain is not effectively treated, the stress and concurrent reflex reactions often cause hypoxia, hypercapnia, hypertension, excessive cardiac activity, and permanent emotional difficulties. The problems associated with these reactions range from prolonged recovery time to death.

Hypersensitivity to painful signals (i.e., sensation of more pain than the stimulus would warrant) a/k/a hyperalgesia can result from persistent pain or from other causes. Similarly, allodynia (i.e., a condition in which ordinarily painless stimuli induce the experience of pain) can also result from a persistent pain condition or not be connected to another pain condition, as can enhanced pain perception and enhanced memory of pain. Hyperalgesia is commonly classified into visceral and somatic hyperalgesia (in turn sometimes divided into musculoskeletal and cutaneous). While visceral hyperalgesia is characterized by altered sensations (e.g., to intraluminal contents) which typically arise in the absence of tissue insult or inflammation, somatic hyperalgesia is usually associated with tissue injury and inflammation. Hyperalgesia may develop and be maintained by either peripheral or central mechanisms. The altered sensations associated with gastroesophageal reflux disease (GERD) or functional gastrointestinal disorders such as functional dyspepsia and irritable bowel syndrome (IBS) are believed to be contributed to by both peripheral and central mechanisms. (reviewed in Gebhart, Am. J. Physiol. Gastrointest. Liver Physiol., 278: G834-G838, 2000)

These types of pain hypersensitivity have been in turn related to (but are not identical to) another type of pain termed “neuropathic pain,” which is a peripheral pain hypersensitivity attributed to a functional disturbance of a nerve, which can occur as a result of alterations (e.g., disease) and/or injury. It can occur by a variety of mechanisms including irritation, injury and compression of the peripheral nerves. The symptoms of neuropathic pain usually include a burning sensation, tingling, or electric-shock-like feelings that may be triggered by even a very light touch.

The best way to manage pain is to treat its cause. However, when such treatment is unavailable or ineffective, or when the cause of the pain is not known, pain-relief methods are used.

The World Health Organization (WHO) recognizes a “Three-step Analgesic Ladder” for pharmacologic management of pain. The ladder begins with relatively low doses of low-potency analgesics and progresses to higher doses of more potent compounds. At present, the three steps involve use of: non-opioid analgesics with or without co-analgesics, such as non-steroid anti-inflammatory drugs (NSAIDs) and cyclooxygenase 2 (COX-2) inhibitors; lower-potency opioids with or without co-analgesics as pain persists or increases to moderate levels; high-potency opioids with or without non-opioid co-analgesics as pain persists or increases to severe levels.

Use of opioid analgesics, even for treatment of severe pain, is controversial in the medical community, due to the possibility of addiction. See, e.g., S. E. Weitz et al., New Jersey Medicine, Vol. 97: 63-67 (2000). Accordingly, additional effective and non-addictive non-opioid analgesics are urgently needed.

Pain is initiated when the peripheral terminals of a subgroup of sensory neurons are activated by noxious chemical, mechanical or thermal stimuli. These neurons, called nociceptors, transmit information regarding tissue damage to pain-processing centers in the spinal cord and brain (Fields, Pain, McGraw-Hill, New York, 1987).

For example, tissue injury results in the production of inflammatory mediators, several of which sensitize primary afferent nociceptors resulting in hyperalgesic pain (i.e., sensation of more pain than the stimulus would warrant). It has been suggested that PGE-2, adenosine, and serotonin-induced hyperalgesia, as well as hyperalgesia induced by tissue damage, are initiated by activation of adenylyl cyclase-cAMP-PKA second messenger cascade. Prolonged hyperalgesia after a sustained exposure to hyperalgesic mediators may result from prolonged exposure to cAMP. Another protein kinase that has been involved in nociceptive pathways mediating epinephrine, bradykinin, NGF, diabetic neuropathy and nerve ligation-induced hyperalgesia is protein kinase C.

Perception of pain can be divided into three areas; acute nociceptive processing, facilitated pain arising from persistent afferent input (as after tissue injury) and neuropathic pain that arises from altered processing after nerve injury.

Nociceptors are unique among sensory neurons because they can be sensitized. The decrease in the threshold and increase in the response to a constant stimulus that are characteristic of nociceptor sensitization are thought to underlie the hyperalgesia or tenderness associated with tissue injury. Agents released at the site of tissue injury sensitize nociceptors by initiating a cascade of events that likely results in a change in ionic conductance of the nociceptor peripheral terminal. A variety of inflammatory insults and direct damage to sensory neuron fibers produce a decrease in the thresholds of activation of sensory neurons, while prolonged activation of sensory neurons can lead to central sensitization to noxious input within the spinal cord.

Neuropathic pain is caused by damage to neural structures, such as damage to peripheral nerve endings or nociceptors, which become extremely sensitive to stimulation and can generate impulses in the absence of stimulation (e.g., herpes zoster pain after the rash has healed). Peripheral nerve damage can lead to pathological states where there is a reduction in pain threshold (i.e., allodynia), an increased response to noxious stimuli (hyperalgesia), or an increased response duration (persistent pain). Goodman & Gilman's The Pharmacological Basis of Therapeutics 529 (Joel G. Hardman et al. eds., 9th ed. 1996); Harrison's Principles of Internal Medicine 53-58 (Anthony S. Fauci et al. eds., 14th ed. 1998).

Spontaneous and/or evoked hyperexcitability of the nerve after injury is considered to be a principal feature of the underlying pathophysiology associated with many chronic, in particular neuropathic, pain syndromes (Devor, M. (1994) in Textbook of Pain, eds. Wall, P. D. & Melzack, R. (Churchill Livingstone, Edinburgh), pp. 79-100; Woolf, C. J., ibid, pp. 101-112). For example, hypersensitivity and hyperexcitability of visceral sensory and visceral motor neurons is associated with irritable bowel syndrome (IBS).

A prominent molecular basis for this abnormal, repetitive firing of injured primary afferents is an accumulation and increased membrane density of sodium channels at focal sites of injury (Devor, M., Govrin-Lippmann, R. & Angelides, K. (1993) J. Neurosci. 13, 1976-1992; England, J. D., Happel, L. T., Kline, D. G., Gamboni, F., Thouron, C. L., Liu, Z. P. & Levinson, S. R. (1996) Neurology 47, 272-276). The resultant membrane remodeling contributes to a lower threshold for action potential generation at these sites and, consequently, precipitates ectopic impulse generation (Wall, P. D. & Gutnick, M. (1974) Nature (London) 248, 740-743; Matzner, O. & Devor, M. J. (1994) J. Neurophysiol. 72, 349-359).

Recent clinical data suggest that chronic pain due to nerve or soft tissue injury may result in the sensitization of the central nervous system, mediated in part by the excitatory amino acids, glutamate and aspartate (Sang, J. Pain Symptom. Manage., 2000, 19(1 Suppl):S21-5). The excessive or pathological activation of glutamate receptors, particularly those that are selectively activated by N-methyl-D-aspartate (NMDA), has been implicated in the processes that underlie pain. It has been demonstrated that hyperalgesia and allodynia following peripheral tissue or nerve injury are not only due to an increase in the sensitivity of primary afferent nociceptors at the site of injury but also depend on NMDA receptor-mediated central changes in synaptic excitability (Parsons, Eur. J. Pharmacol., 2001, 429(1-3):71-8). Functional inhibition of NMDA receptors can be achieved through actions at different recognition sites such as the primary transmitter site (competitive), strychnine-insensitive glycine site (glycine(B)), polyamine site (NR2B selective) and phencyclidine site located inside the cationic channel (Kleckner and Dingledine, Science, 1988, 241:835-837; McBain et al., Mol. Pharmacol., 1989, 36:556-565; Danysz and Parsons, Pharmacol. Rev., 1998, 50:597-664).

Unfortunately, most agents which completely block NMDA receptors cause numerous side effects such as memory impairment, psychotomimetic effects, ataxia and motor incoordination. Thus, in the recent studies employing clinically available ketamine and dextromethorphan (NMDA-receptor antagonists with affinity at the phencyclidine site), they have been shown to modulate pain and hyperalgesia but were limited by dose-limiting side effects. Clinical trials also failed to support good therapeutic utility due to numerous side effects for such NMDA receptor antagonists as Dizocilpine ((+)MK-801; (+)-5-methyl-10,11-dihydro-5H-dibenzocyclohepten-5,10-imine maleate), Cerestat (CNS-1102), Licostinel (ACEA 1021), Selfotel (CGS-19755), and D-CPP-ene (Leppik, Epilepsia, 1998, 39 (Suppl 5):2-6; Sveinbjornsdottir et al, Epilepsia, 1993, 34:493-521; SCRIP 2229/30, 1997, p. 21). It has therefore been a challenge in the field to develop NMDA receptor antagonists that prevent the pathological activation of NMDA receptors but still allow their physiological activity.

There is now considerable evidence that moderate affinity channel blockers, glycine(B) and NR2B selective antagonists show a much better profile in animal models than high affinity channel blockers and competitive NMDA receptor antagonists. These “therapeutically” safe NMDA receptor antagonists are also able to slow or prevent the development of opioid tolerance, indicating the utility of their combination with opioids in the treatment of pain. Peripheral NMDA receptors offer a very attractive target for NMDA receptor antagonists that do not cross the blood brain barrier in inflammatory and visceral pain. Such agents might be predicted to be devoid of serious central nervous system (CNS) side effects at doses producing powerful antinociception at peripheral NMDA receptors.

Memantine (1-amino-3,5-dimethyl adamantane; disclosed, e.g., in U.S. Pat. Nos. 4,122,193; 4,273,774; 5,061,703) is such a noncompetitive NMDA receptor inhibitor, which is clinically available and has been implicated in alleviating, among many other diseases, neuropathic pain (U.S. Pat. No. 5,334,618) and inflammatory induced pain (U.S. Pat. No. 6,221,887). Thus, relatively high doses of memantine were shown to selectively block thermal hyperalgesia and mechanical allodynia in some models of chronic and neuropathic pain without obvious effects on motor reflexes.

Neramexane (1-amino-1,3,3,5,5-pentamethylcyclohexane) is another derivative of 1-amino-cyclohexane (disclosed, e.g., in U.S. Pat. Nos. 6,034,134 and 6,071,966) within a subclass devoid of an adamantane (pyramidal) structure.

Memantine, neramexane as well as some other 1-amino-alkylcyclohexanes are systemically-active noncompetitive NMDA receptor antagonists having moderate affinity for the receptor. They exhibit strong voltage dependent characteristics and fast blocking/unblocking kinetics (Parsons et al., 1999, supra; Görtelmeyer et al., Arzneim-Forsch/Drug Res., 1992, 42:904-913; Winblad et al., Int. J. Geriat. Psychiatry, 1999, 14:135-146; Rogawski, Amino Acids, 2000, 19: 133-49; Danysz et al., Curr. Pharm. Des., 2002, 8:835-43; Jirgensons et. al., Eur. J. Med. Chem., 2000, 35: 555-565). These compounds dissociate from the NMDA receptor channels much more rapidly than the high affinity NMDA receptor antagonists such as (+)MK-801 and attenuate disruption of neuronal plasticity produced by tonic overstimulation of NMDA receptors probably by causing an increase of the signal-to-noise ratio. Due to their relatively low affinity for the receptor, strong voltage dependency and fast receptor unblocking kinetics, these compounds are essentially devoid of the side effects of other NMDA receptor antagonists at doses within the therapeutic range Kornhuber et al., Eur. J. Pharmacol., 1991, 206:297-311). Indeed, memantine has been applied clinically for over 15 years showing good tolerability with the number of treated patients exceeding 200,000 (Parsons et al., 1999, supra).

1-Amino-cyclohexane derivatives such as memantine and neramexane (see U.S. patent application Ser. No. 09/597,102 and its corresponding international patent application published as WO 01/98253; U.S. Pat. No. 6,034,134) have also been suggested to function via non-NMDA-mediated pathways. Thus, memantine was shown to inhibit 5HT3-mediated current (in the native N1E-115 and heterologous HEK-293 cells) and NMDA receptor-mediated currents (in rat hippocampal slices) with approximately equal affinity (Parsons et al., 1999, supra; Rammes et al., 2001, Neurosci. Lett., 306:81-84).

In light of the above, there is still a need in the art to develop noncompetitive NMDA receptor antagonists for the treatment of pain hypersensitivity. There is also a need in the art to develop noncompetitive NMDA receptor antagonists, other than memantine, for the treatment of neuropathic pain.

The present invention satisfies these and other needs by disclosing for the first time that 1-amino-alkylcyclohexane derivatives such as neramexane (which are not adamantane derivatives like memantine) are useful for the treatment of pain hypersensitivity and neuropathic pain.

### SUMMARY OF THE INVENTION

The instant invention provides a novel method useful for treating pain hypersensitivity in a mammal, said method comprising administering to the mammal an 1-amino-alkylcyclohexane derivative in amounts effective for this purpose. The pain hypersensitivity disorder can be visceral hypersensitivity disorder such as functional dyspepsia, irritable bowel syndrome (IBS), and gastroesophageal reflux disease (GERD), or any hyperalgesia or allodynia whether it is co-incident with another pain condition or not.

The instant invention further provides a novel method useful for treating neuropathic pain in a mammal, said method comprising administering to the mammal an 1-amino-alkylcyclohexane derivative in amounts effective for this purpose.

In a specific embodiment, the 1-aminocyclohexane derivative useful in the methods of the invention is neramexane. Preferably, the mammal is human. Also preferably, the 1-amino-alkylcyclohexane derivatives are administered in therapeutically effective dosages. For memantine, such therapeutically effective dosages are preferably in the range of 1-200 mg/day; most preferably, in the range of 5-80 mg/day and especially 10-40 mg/day. For neramexane, such therapeutically effective dosages are preferably in the range of 1-200 mg/day; most preferably, in the range of 5-60 mg/day and especially 10-40 mg/day. Therapeutically effective dosages for human use of neramexane are preferably in the range of 5-100 mg/human/day; most preferably, in the range of 12.5-100 mg/human/day and especially 12.5-80 mg/human/day.

In conjunction with the methods of the present invention, also provided herein are pharmaceutical compositions comprising therapeutically effective amounts of a non-adamantane 1-amino-alkylcyclohexane derivative (preferably, neramexane) and, optionally, at least one pharmaceutically acceptable carrier or excipient.

### DETAILED DESCRIPTION OF THE INVENTION

The instant invention provides novel methods useful for treating pain hypersensitivity and/or neuropathic pain in a mammal, said method comprising administering to the mammal a particular 1-amino-alkylcyclohexane derivative in amounts effective for this purpose. Accordingly, the present invention provides methods for treating hypersensitivity-related pain disorders such as visceral hypersensitivity disorders, neuropathic pain, as well as allodynia, and hyperalgesia associated with cancer related pain, migraine, osteoarthritis, and rheumatoid arthritis. Visceral hypersensitivity disorders treatable by the method of the present invention include gastroesophageal reflux disease (GERD), gastritis, all functional pediatric disorders and all functional gastrointestinal disorders including but not limited to irritable bowel syndrome (IBS) including irritable bowel disease (IBD), functional dyspepsia (for example, ulcer-like dyspepsia, dysmotility-like dyspepsia, functional heartburn, and non-ulcer dyspepsia), functional chest pain of presumed oesophageal origin, functional dysphagia, non-cardiac chest pain, symptomatic gastro-oesophageal disease, aerophagia, functional constipation, functional diarrhea, chronic functional abdominal pain, recurrent abdominal pain (RAP), functional abdominal bloating, functional biliary pain, functional incontinence, functional ano-rectal pain, chronic pelvic pain, pelvic floor dyssenergia, un-specified functional ano-rectal disorder, cholecystalgia, interstitial cystitis, dysmenorrhea, and dyspareunia.

In a preferred embodiment, the 1-aminocyclohexane derivative useful in the method of the invention is neramexane. Preferably, the mammal is human. Also preferably, the 1-amino-alkylcyclohexane derivatives are administered in therapeutically effective dosages, which are in the range 1-200 mg/day; most preferably, in the range 5-60 mg/day and especially at 10-40 mg/day.

In conjunction with the methods of the present invention, also provided herein are pharmaceutical compositions comprising therapeutically effective amounts of a non-adamantane 1-amino-alkylcyclohexane derivative (preferably, neramexane) and, optionally, at least one pharmaceutically acceptable carrier or excipient.

The compositions and methods of the invention can be used to treat pain, hypersensitivity such as that resulting from noxious hyperstimulation of peripheral nociceptors. The compositions and methods of the invention can be used to treat pain hypersensitivity whether it is known to be related to or induced by a disease, trauma, or another tissue or neuronal injury or any nociceptor hypersensitization.

### Definitions

As used herein, the term “pain” is art recognized and includes a bodily sensation elicited by noxious chemical, mechanical, or thermal stimuli, in a subject, e.g. a mammal such as a human. The term “pain” includes chronic pain such as lower back pain; pain due to arthritis, e.g., osteoarthritis; joint pain, e.g., knee pain or carpal tunnel syndrome; myofascial pain, and neuropathic pain. The term “pain” further includes acute pain, such as pain associated with muscle strains and sprains; tooth pain; headaches; pain associated with surgery; or pain associated with various forms of tissue injury, e.g., inflammation, infection, and ischemia. The term “pain” also includes central pain as well as all kinds pain hypersensitivity and neuropathic as defined below.

The term “pain hypersensitivity” is used herein to refer to hypersensitivity to painful signals (i.e., sensation of more pain than the stimulus would warrant) a/k/a hyperalgesia, allodynia (i.e., a condition in which ordinarily painless stimuli induce the experience of pain), enhanced pain perception, and enhanced memory of pain. As used herein, the term “pain hypersensitivity” encompasses both visceral and somatic hyperalgesia. As used herein in connection with various visceral disorders, the term “pain hypersensitivity” therefore generally refers to visceral hyperalgesia, which is characterized by altered sensations (e.g., to intraluminal contents) which typically arise in the absence of tissue insult or inflammation, in contrast to somatic hyperlagesia, which is commonly associated with tissue injury and inflammation.

The term “neuropathic pain” is used herein to refer to a peripheral pain hypersensitivity attributed to a functional disturbance of a nerve, which can occur as a result of alterations (e.g., disease) and/or injury. It can occur by a variety of mechanisms including irritation, injury and compression of the peripheral nerves. The symptoms of neuropathic pain usually include a burning sensation, tingling, or electric-shock-like feelings that may be triggered by even a very light touch.

The term “treat” is used herein to mean to relieve or alleviate pain in a hypersensitive mammal or in a mammal suffering from neuropathic pain. The term “treat” may mean to relieve or alleviate the intensity and/or duration of a pain (e.g., burning sensation, tingling, electric-shock-like feelings, etc.) experienced by a subject in response to a given stimulus (e.g., pressure, tissue injury, cold temperature, etc.).

Within the meaning of the present invention, the term “NMDA antagonist drugs” is used to refer to drugs, that can suppress the normal triggering of NMDA receptor-mediated neuronal firings. Preferred NMDA antagonist drugs of the invention are 1-amino-alkylcyclohexane derivatives such as neramexane. These compounds may also have 5HT₃ antagonist activity and/or neuronal nicotinic receptor antagonist activity.

The term “1-amino-alkylcyclohexane derivative” is used herein to describe a compound which is derived from 1-amino-alkylcyclohexane (or an available derivative thereof, such as neramexane) in the process used to create a similar but slightly different drug.

The 1-amino-alkylcyclohexane derivatives of the present invention can be represented by the general formula (I): 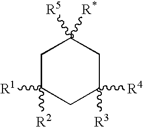
wherein R* is —(CH₂)ₙ—(CR⁶R⁷)ₘ—NR⁸R⁹
wherein n+m=0, 1, or 2
wherein R¹ through R⁷ are independently selected from the group consisting of hydrogen and lower-alkyl (1-6C), at least R¹, R⁴, and R⁵ being lower-alkyl, and wherein R⁸ and R⁹ are independently selected from the group consisting of hydrogen and lower-alkyl (1-6C) or together represent lower-alkylene —(CH₂)ₓ— wherein x is 2 to 5, inclusive, and enantiomers, optical isomers, hydrates, and pharmaceutically-acceptable salts thereof.

Non-limiting examples of 1-amino-alkylcyclohexane derivatives used according to the invention are selected from the group consisting of:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane,
N-ethyl-1-amino-1,3,3,5,5-pentamethyl-cyclohexane,
N-(1,3,3,5,5-pentamethylcyclohexyl)pyrrolidine,
3,3,5,5-tetramethylcyclohexylmethylamine,
1-amino-1-propyl-3,3,5,5-tetramethylcyclohexane,
1 amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3(cis)-propylcyclohexane,
1-amino-1,3-dimethyl-3-ethylcyclohexane,
1-amino-1,3,3-trimethylcyclohexane,
cis-3-ethyl-1(trans)-3(trans)-5-trimethylcyclohexamine,
1-amino-1,3(trans)-dimethylcyclohexane,
1,3,3-trimethyl-5,5-dipropylcyclohexylamine,
1-amino-1-methyl-3(trans)-propylcyclohexane,
1-methyl-3(cis)-propylcyclohexylamine,
1-amino-1-methyl-3(trans)-ethylcyclohexane,
1-amino-1,3,3-trimethyl-5(cis)-ethylcyclohexane,
1-amino-1,3,3-triethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-1-amino-1,3,3,5,5-pentamethylcyclohexane,
1-amino-1-methylcyclohexane,
N,N-dimethyl-1-amino-1,3,3,5,5-pentamethylcyclohexane,
2-(3,3,5,5-tetramethylcyclohexyl)ethylamine,
2-methyl-1-(3,3,5,5-tetramethylcyclohexyl)propyl-2-amine,
2-(1,3,3,5,5-pentamethylcyclohexyl-)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,SS)trans-5-ethyl-1,3,3-trimethylcyclohexane,
1-ammo-(1S,SS)cis-5-ethyl-1,3,3-trimethylcyclohexane,
1-amino-1,5,5-trimethyl-3(cis)-isopropyl-cyclohexane,
1-amino-1,5,5-trimethyl-3(trans)-isopropyl-cyclohexane,
1-amino-1-methyl-3(cis)-ethyl-cyclohexane,
1-amino-1-methyl-3(cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-1-ethyl-3,3,5,5-tetramethylcyclohexane,
N-ethyl-1-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,SS)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,SS)trans-5-ethyl,3,3-trimethylcyclohexyl]pyrrolidine or piperidine,
N-(1-ethyl-3,3,5,5-tetramethylyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,
   their optical isomers, diastereomers, enantiomers, hydrates, their pharmaceutically acceptable salts, and mixtures thereof.

Neramexane (1-amino-1,3,3,5,5-pentamethylcyclohexane) is disclosed, e.g., in U.S. Pat. Nos. 6,034,134 and 6,071,966. For details on synthesis see U.S. Pat. No. 6,034,134. Additional synthetic techniques for the foregoing compounds can be found in provisional applications Ser. No. 60/350,974 filed Nov. 7, 2001, Ser. No. 60/337,858 filed Nov. 8, 2001, and Ser. No. 60/366,386 filed Mar. 21, 2002, all incorporated by reference.

According to the invention, the 1-amino-alkylcyclohexane derivatives of formula (I) may be applied as such or used in the form of their pharmaceutically-acceptable salts including, for example, the acid addition salts such as hydrochlorides, hydrobromides, sulfates, acetates, succinates or tartrates, or their acid addition salts with fumaric, maleic, citric, or phosphoric acids.

Various salts and isomers (including stereoisomers and enantiomers) of the drugs listed herein can be used. The term “salts” can include addition salts of free acids or free bases. Examples of acids which may be employed to form pharmaceutically acceptable acid addition salts include inorganic acids such as hydrochloric, sulfuric, or phosphoric acid, and organic acids such as acetic, maleic, succinic, or citric acid, etc. All of these salts (or other similar salts) may be prepared by conventional means. The nature of the salt or isomer is not critical, provided that it is non-toxic and does not substantially interfere with the desired pharmacological activity.

The term “therapeutically effective” applied to dose or amount refers to that quantity of a compound or pharmaceutical composition that is sufficient to reduce or eliminate pain hypersensitivity. The reduction can be assessed by measuring intensity or duration or both of hypersensitive pain (e.g., burning sensation, tingling, electric-shock-like feelings, etc.) experienced by the mammal in response to a given stimulus (e.g., pressure, tissue injury, cold temperature, etc.).

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The term “carrier” applied to pharmaceutical compositions of the invention refers to a diluent, excipient, or vehicle with which an 1-amino-alkylcyclohexane derivative is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18^{th} Edition.

The term “subject” as used herein refers to a mammal (e.g., rodent such as mouse or rat). In particular, the term refers to humans.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

### Animal Models of Pain and Testing Methods

A common test in experimental animals for the peripheral analgesic activity in situations of acute hypersensitivity and hyperreactivity consists of first inducing a local irritation via intraperitoneal (i.p.) injection of an agent (e.g., acetic acid), and then provoking the pain by mechanical distention. After acetic acid injection, the animal twists its body around, especially the abdominal wall which undergoes contractions, whence the name “writhing test” commonly used for this test. In the version proposed by R. Koster (Fed. Proc. 1959, 18, p. 412) the test drugs are administered orally ten minutes before the i.p. injection of the acetic acid solution. A modification proposed by G. A. Bentley (Br. J. Pharm. 1981, 73, pp. 325-332) consists in administering the test drugs via the i.p. route a few minutes after the administration of the acid solution. This modification shows up the immediate local antinociceptive action of the compounds. The percentage activity for each dose of a test compound administered is calculated and compared with the control group, and the results are expressed as an ED₅₀, namely the dose required to inhibit 50% of the abdominal cramps induced by the administration of acetic acid under the conditions of the experiment.

A chronic hyperalgesic condition can be reproduced in mice or rats by the injection of Freund's complete adjuvant (FCA) containing heat-killed Mycobacterium butyricum into the lower lumbar region or directly into the hind footpads (Colpaert et al., Life Sci., 27:921-928, 1980; De Castro Costa et al., Pain, 10:173-185, 1981; Larson et al., Pharmacol. Biochem Behav., 24:49-53, 1986). Although little or no visible inflammation is observed after a single intradermal injection of FCA, significant alterations in nociceptive thresholds occur. These alterations may be measured by decreases in response latency (compared to animals injected with the same adjuvant lacking heat-killed Mycobacterium butyricum) in tail-flick response to a radiant heat source (D'Amour and Smith, J. Pharmacol. Exp. Ther., 72:74-79, 1941) or hot-plate assay involving placing animals on a hot plate maintained, e.g., at 52.5° (Eddy and Leimbach, J. Pharmacol. Exp. Ther., 107:385-393, 1953). In each test, the response latency is measured as the time preceding licks of a hindpaw and a forepaw. In this animal model, changes in the response latency to a noxious stimulus in the areas surrounding the inflamed tissue are similar to those observed in non-inflamed tissue far from the site of injection, suggesting that changes in sensitivity to noxious stimuli are not merely the result of local hypersensitivity of the inflamed tissue, but may also be due to alterations in nociception at the level of the central nervous system (CNS).

One of the most commonly used experimental animal models for neuropathic pain and pain hypersensitivity is the chronic constriction injury (CCI) model where four loose ligatures are tied around the sciatic nerve (Bennett and Xie, Pain, 33:87-107, 1988). One disadvantage of this model is the introduction of foreign material into the wound, which causes a local inflammatory reaction, whereas hyperalgesia does not have to be associated with inflammation. Thus the distinction between the neuropathic and the inflammatory component of pain is difficult in this model. In order to produce a pure nerve injury model without an epineurial inflammatory component due to foreign material, Lindenlaub and Sommer (Pain, 89:97-106, 2000) have recently performed a partial sciatic nerve transection (PST) in rats. These rats developed thermal hyperalgesia and mechanical allodynia comparable to the CCI model. PST model is considered by some researches to provide a better evaluation of pain hypersensitivity. In both models, animals' thermal and mechanical withdrawal thresholds are assessed before and after surgery. Thermal withdrawal is commonly assessed by response to radiant heat on the planar surface of the hindpaw (Hargreaves et al., Pain, 32:77-88, 1988). Mechanical hypersensitivity is commonly determined by measuring the withdrawal thresholds to von Frey hairs (Stoelting; see Dixon, J. Am Stat. Assoc., 60:967-978, 1965).

CCI and PST models involve acute or subacute insult of the peripheral nerve, and do not necessarily reflect gradual but progressive insult of the nerve, which is expected to occur in such common neuropathic pain conditions as neuropathic cancer pain. Neuropathic cancer pain can be, however, reproduced by inoculating Meth A sarcoma cells to the immediate proximity of the sciatic nerve in BALB/c mice (Shimoyama et al., Pain, 99:167-174, 2002). The tumor grows predictably with time and gradually compresses the nerve, thereby causing thermal hyperalgesia (as determined by paw withdrawal latencies to radiant heat stimulation), mechanical allodynia (as determined by sensitivity of paws to von Frey hairs), and signs of spontaneous pain (as detected by lifting of the paw).

A human surrogate model of neuropathic pain and peripheral hypersensitivity based on intradermal capsaicin injection is disclosed in detail in Example 1, infra.

In human patients, visceral hypersensitivity is characterized by decreased pain and sensation thresholds to distension. Accordingly, visceral hypersensitivity is usually appraised by measurement of threshold volumes or pressures for first sensation of pain or by increased scores of symptoms (including pain) in response to standard stimuli. Specifically, visceral hypersensitivity may be tested via intubation of the viscus of interest and application of mechanical stimuli such as balloon distension with monitoring of either perseption scores on a visual analogue scale (VAS), threshold perceptions, or changes in cerebral blood flow (Camilleri, Gut, 51: (Suppl. 1):i34-i40, 2002). A liquid nutrient or non-nutrient test has been also developed to identify patients with hypersensitivity due to functional dyspepsia (Tack et al., Gastroenterology, 115:1346-1352, 1998; Tosetti et al., Gastroenterology, 116:A336, 1999). In this test, the combination of volume measurements with a non-nutrient drink test and measurement of symptoms such as satiety, pain nausea, fullness, and bloating 30 minutes after ingestion of the maximum volume of nutrient or non-nutrient liquid provides a clinically applicable means to assess both accommodation and sensation responses (Kim et al., Am. J. Gastroenterol., 96:3099-3105, 2001).

### Pharmaceutical Compositions

In conjunction with the methods of the present invention, also provided are pharmaceutical compositions comprising a therapeutically effective amount of an 1-amino-alkylcyclohexane derivative (such as neramexane) as well as, optionally, an additional carrier or excipient (all pharmaceutically acceptable). The compositions can be formulated for once-a-day administration or several-times-a-day administration.

In the disclosed compositions, preferably, the 1-amino-alkylcyclohexane derivative is present in therapeutically effective amounts. The optimal therapeutically effective amount should be determined experimentally, taking into consideration the exact mode of administration, form in which the drug is administered, the indication toward which the administration is directed, the subject involved (e.g., body weight, health, age, sex, etc.), and the preference and experience of the physician or veterinarian in charge. As disclosed herein, for human administration, the 1-amino-alkylcyclohexane derivatives are administered in suitable form in doses ranging from about 1-200 mg per day; preferably in doses ranging 5-80 mg/day, and especially 10-40 mg/day. It may also be desirable in certain cases to administer the active ingredients in a suboptional or subthreshold amount, and such administration would also be within the invention.

### Administration

The active agents of the present invention may be administered orally, topically, parenterally, or mucosally (e.g., buccally, by inhalation, or rectally) in dosage unit formulations containing conventional non-toxic pharmaceutically acceptable carriers. It is usually desirable to use the oral or topical route.

The active agents may be administered orally in the form of a capsule, a tablet, or the like (see Remington's Pharmaceutical Sciences, Mack 5 Publishing Co., Easton, Pa.). The orally administered medicaments may be administered in the form of a time-controlled release vehicle, including diffusion-controlled systems, osmotic devices, dissolution-controlled matrices, and erodible/degradable matrices.

For oral administration in the form of a tablet or capsule, the active drug component can be combined with a non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like. For oral administration in liquid form, the drug components can be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms.

The tablets can be coated by methods well known in the art. The compositions of the invention can be also introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO95/11010 and WO93/07861). Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound. A particular example of an oral time-controlled release pharmaceutical formulation is described in U.S. Pat. No. 5,366,738.

The formulations of the invention can be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. The compositions can take such forms as excipients, suspensions, solutions, or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use.

Compositions of the present invention can also be formulated for rectal administration, e.g., as suppositories or retention enemas (e.g., containing conventional suppository bases such as cocoa butter or other glycerides).

Although the active agents of the present invention may be administered in divided doses, for example, two or three times daily, a single daily dose of the 1-amino-alkylcyclohexane derivative is preferred. Such a dose may be preferably achieved by a modified release formulation. Such formulations are well-known.

Preferred specific amounts of the 1-amino-alkylcyclohexane derivative which may be used in unit dosage amounts of the invention include, for example, 5 mg, 10 mg, 15 mg, and 20 mg for memantine and 5 mg, 10 mg, 20 mg, 30 mg, and 40 mg for neramexane.

Fine-tuning of the administered dose may be achieved as described below.

Effective doses and toxicity of the compounds and compositions of the instant invention, which performed well in in vitro tests, can be determined or fine-tuned in preclinical studies using small animal models (e.g., mice or rats) in which the 1-amino-alkylcyclohexane derivatives have been found to be therapeutically effective and in which these drugs can be administered by the same route proposed for the human clinical trials. Preferred animal models of the invention are disclosed in the section entitled “Animal Models of Pain and Testing Methods”, supra.

For any pharmaceutical composition used in the methods of the invention, the therapeutically effective dose can be estimated initially from animal models to achieve a circulating plasma concentration range that includes the IC₅₀ (i.e., the concentration of the test compound which achieves a half-maximal inhibition of pain hypersensitivity or neuropathic pain). Dose-response curves derived from animal systems are then used to determine testing doses for the initial clinical studies in humans. In safety determinations for each composition, the dose and frequency of administration should meet or exceed those anticipated for use in the clinical trial.

A specific dose naturally varies depending on the dosage procedure, the conditions of a patient or a subject animal such as age, body weight, sex, sensitivity, feed, dosage period, drugs used in combination, seriousness of the disease. The appropriate dose and dosage times under certain conditions can be determined by the test based on the above-described indices but may be refined and ultimately decided according to the judgment of the practitioner and each patient's circumstances (age, general condition, severity of symptoms, sex, etc.) according to standard clinical techniques. As disclosed herein, an appropriate human dose of an 1-amino-alkylcyclohexane derivative such as neramexane is generally in the range of 5-100 mg/human/day, preferably in the range of 12.5-100 mg/human/day, most preferably in the range 12.5-80 mg/human/day.

Toxicity and therapeutic efficacy of the compositions of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀. Compositions that exhibit large therapeutic indices are preferred.

The data obtained from animal studies can be used in formulating or refining a range of doses for use in humans. The therapeutically effective doses of 1-amino-alkylcyclohexane derivatives in humans lay preferably within a range of circulating concentrations that include the ED₅₀ with little or no toxicity. For example, such a therapeutically effective circulating concentration for neramexane is 1 μM. The dosage can vary within this range depending upon the dosage form employed and the route of administration utilized and the pharmacokinetics of the active ingredient. Ideally, a single dose of the drug should be used daily.

The pharmaceutical compositions of the invention are not only highly effective at relatively low doses but also possess low toxicity and produce few side effects. Indeed, the only common side effect for the 1-amino-alkylcyclohexane derivatives of the invention is a minor motor and cognitive impairment (reflected, e.g., in nausea, vomiting, dizziness, or confusion).

### EXAMPLES

### Example 1

### Analgesic and Antihyperalgesic Properties of the NMDA Receptor Antagonist Neramexane in a Human Surrogate Model of Neuropathic

### Pain (Intradermal Capsaicin Injection)

The experimentally induced facilitation of pain perception adjacent to an injury of the skin (secondary hyperalgesia) is a well characterized human model of central sensitization of the nociceptive system, which displays intriguing similarities with the hyperalgesia subtype of neuropathic pain (Baumgartner et al. Pain 96 (2002) 141-151). This surrogate model of neuropathic pain is thus a valid method for early clinical testing of substances for the treatment of neuropathic pain. The efficacy of the neramexane, a moderate affinity, uncompetitive NMDA receptor antagonist, was tested in a placebo-controlled double-blind crossover study in the intradermal capsaicin injection model.

Capsaicin (40 μg) was injected intradermally in the skin of the ventral forearm 3 hours after administration of a single oral dose of neramexane hydrochloride (40 mg) or placebo. Changes of pain sensitivity adjacent to the capsaicin injection and in a remote control area were tested in parallel by quantitative sensory testing prior to oral neramexane or placebo, 2½ hours after neramexane or placebo (i.e., prior to capsaicin injection) and 1½ hours after capsaicin injection.

The capsaicin injection elicited a strong, over 5-10 min rapidly declining burning pain resulting in enhanced pain sensitivity adjacent to the injection including hyperalgesia to pin prick and pain to light touch (“allodynia”).

The capsaicin-induced burning pain was significantly reduced by 21% after neramexane compared to placebo during the first minute after injection (p<0.01) and remained lower by 33% in the following minutes (2^{nd}-5^{th} min; n.s., due to increasing variability). The size of the capsaicin-induced axon reflex erythema remained unaltered.

Treatment with neramexane significantly reduced the pain of noxious pin pricks at any time in the control area, and additionally in the test area before capsaicin injection by 27-31% compared to placebo (p<0.001; analgesia). The reduction of pin prick pain was even greater in the test area after capsaicin injection (39% compared to placebo, p<0.001; combined analgesia and antihyperalgesia). Antihyperalgesia alone, as calculated from the ratio of test and control area (9%), was not significant.

It is believed that because neramexane has an analgesic effect, the test showed only a trend rather than a statistically significant result. In other words, the analgesic effect of neramexane blunts the capsaicin-induced pain which in turn reduces the hypersensitivity. That this is the case is also supported by the allodynia results below.

Pain to stroking light touch stimuli (“allodynia”) was significantly reduced after neramexane by 22% across all test times (p=0.07), and by 25% during the first 30 min after capsaicin injection (p<0.05). This result is unexpected.

Summation of pain to repetitive pin prick stimulation (trains of 10 stimuli at 1 Hz) at baseline (prior to neramexane or placebo) was characterized by approximately a doubling of the pain during the plateau of the last stimuli compared to the first one. However, summation of pain was never changed after neramexane or placebo.

The combined effect of a strong analgesic along with a mild antihyperalgesic action (evidenced by the 9% trend above) shows that the NMDA receptor antagonist neramexane is of value for the treatment of neuropathic pain, or pain hypersensitivity and conditions related to pain hypersensitivity.

### Example 2

### Large-Scale Clinical Studies in Humans Suffering from Pain Hypersensitivity

A protocol requires that no other analgesic drugs be taken for 24 hours before participating in the study. Nor are any other analgesic drugs taken during the 3-day period of the use of neramexane. Pain hypersensitivity is initially evaluated prior to the initial neramexane administration. After each administration of neramexane, the intensity of pain hypersensitivity of every patient is observed and recorded at the following several time intervals (e.g., 5 min, 10 min, 15 min, 20 min, 30 min, 1 h, 2 h, 3 h, 4 h, 5 h, 6 h, 7 h, 8 h, and 12 h). Pain hypersensitivity is evaluated using the 0A10 Numeric Pain Intensity Scale recommended by the World Health Organization (WHO). Briefly, patients self-evaluate their pain hypersensitivity based upon a 0 to 10 numeric scale (0=no pain; 1-4=mild pain; 5-6=moderate pain; 7-10=severe pain). Analgesic effect is further determined by calculating Pain Intensity Difference (PID) by taking the intensity number of pain hypersensitivity before administration, and subtracting the intensity number of pain hypersensitivity at every time point after administration. After calculating the intensity of patient's pain hypersensitivity at each time interval, the pain hypersensitivity relief is assessed and assigned a value from one of the following five choices: 0=no relief; I=mild relief (the pain hypersensitivity abates about 25%); II=moderate relief (the pain hypersensitivity abates about 50%); III=significant relief (the pain hypersensitivity abates about 75%); IV=complete relief (the pain hypersensitivity disappears completely).

Analgesic effect of neramexane in pain hypersensitivity is further determined by evaluating the patients' quality of life. Pain hypersensitivity affects every patient's normal life and ability to continue with their everyday routine. This is generally referred to as their quality of life. Depending on the severity of the pain hypersensitivity that is being experienced by each individual it can cause patients to experience irritability, depression and poor appetite. Any changes in the patient's quality of life must be considered, in the evaluation of the analgesic effect of any new drug. It should be noted that this “quality of life” evaluation is a subjective issue which depends on the patient's descriptions of any changes (before and after administration of neramexane) in their quality of life as the primary means of input for making this evaluation. Issues that are questioned in regard to the quality of life include, routine daily activity, emotions, mobility (walking ability), normal work (includes both work outside the home and housework), sleeping state or pattern, relations with other persons, enjoyment of life. Quality of life is evaluated by subjects themselves before and every 8 hours after administration. The quality of life includes mood, walking ability, normal work (includes both work outside the home and housework), and relations with other people, sleep, and enjoyment of life. The numeric scale used to express the extent of pain hypersensitivity interference with the quality of life is as follows: 0=does not interfere; 1-3=mildly interferes; 4-7=moderately interferes; 8-9=severely interferes; 10=completely interferes.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1-24. (canceled)

25. A method for treating pain hypersensitivity in a living animal body, including a human, such method comprising administering to the living animal body, including a human, a therapeutically effective amount of an 1-amino-alkylcyclohexane derivative.

26. The method of claim 25, wherein the pain hypersensitivity is hyperalgesia.

27. The method of claim 25, wherein the pain hypersensitivity is allodynia.

28. The method of claim 25, wherein the pain hypersensitivity is selected from visceral hypersensitivity, musculoskeletal allodynia/hyperalgesia, and cutaneous allodynia/hyperalgesia.

29. The method of claim 28, wherein the visceral hypersensitivity is associated with disorders selected from irritable bowel syndrome (IBS), gastroesophageal reflux disease (GERD), and functional dyspepsia.

30. The method of claim 25, wherein the 1-amino-alkylcyclohexane derivative is selected from those of formula (I): 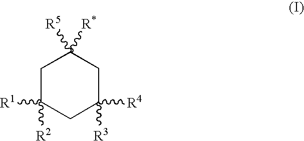
wherein R* is —(CH₂)ₙ—(CR⁶R⁷)ₘ—NR⁸R⁹
wherein n+m=0, 1, or 2
wherein R¹ through R⁷ are independently selected from hydrogen and lower-alkyl (1-6C), at least R¹, R⁴, and R⁵ being lower-alkyl, and wherein R⁸ and R⁹ are independently selected from the group consisting of hydrogen and lower-alkyl (1-6C) or together represent lower-alkylene —(CH₂)ₓ— wherein x is 2 to 5, inclusive, and enantiomers, optical isomers, hydrates, and pharmaceutically-acceptable salts thereof.

31. The method of claim 30, wherein the 1-amino-alkylcyclohexane derivative is selected from:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane,
N-ethyl-1-amino-1,3,3,5,5-pentamethyl-cyclohexane,
N-(1,3,3,5,5-pentamethylcyclohexyl)pyrrolidine,
3,3,5,5-tetramethylcyclohexylmethylamine,
1-amino-1-propyl-3,3,5,5-tetramethylcyclohexane,
1 amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3 (cis)-propylcyclohexane,
1-amino-1,3-dimethyl-3-ethylcyclohexane,
1-amino-1,3,3-trimethylcyclohexane,
cis-3-ethyl-1(trans)-3 (trans)-5-trimethylcyclohexamine,
1-amino-1,3(trans)-dimethylcyclohexane,
1,3,3-trimethyl-5,5-dipropylcyclohexylamine,
1-amino-1-methyl-3 (trans)-propylcyclohexane,
1-methyl-3 (cis)-propylcyclohexylamine,
1-amino-1-methyl-3 (trans)-ethylcyclohexane,
1-amino-1,3,3-trimethyl-5(cis)-ethylcyclohexane,
1-amino-1,3,3-trimethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-1-amino-1,3,3,5,5-pentamethylcyclohexane,
1-amino-1-methylcyclohexane,
N,N-dimethyl-1-amino-1,3,3,5,5-pentamethylcyclohexane,
2-(3,3,5,5-tetramethylcyclohexyl)ethylamine,
2-methyl-1-(3,3,5,5-tetramethylcyclohexyl)propyl-2-amine,
2-(1,3,3,5,5-pentamethylcyclohexyl-1)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,SS)trans-5-ethyl-1,3,3-trimethylcyclohexane,
1-amino-(1S,SS)cis-5-ethyl-1,3,3-trimethylcyclohexane,
1-amino-1,5,5-trimethyl-3(cis)-isopropyl-cyclohexane,
1-amino-1,5,5-trimethyl-3 (trans)-isopropyl-cyclohexane,
1-amino-1-methyl-3 (cis)-ethyl-cyclohexane,
1-amino-1-methyl-3 (cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-1-ethyl-3,3,5,5-tetramethylcyclohexane,
N-ethyl-1-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,SS)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,SS)trans-5-ethyl,3,3-trimethylcyclohexyl]pyrrolidine or piperidine,
N-(1-ethyl-3,3,5,5-tetramethylyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,
their optical isomers, diastereomers, enantiomers, hydrates, their pharmaceutically acceptable salts, and mixtures thereof.

32. A method for treating neuropathic pain in a living animal body, including a human, such method comprising administering to the living animal body, including a human, a therapeutically effective amount of an 1-amino-alkylcyclohexane derivative devoid of an adamantane (pyramidal) structure.

33. The method of claim 30, wherein the 1-amino-alkylcyclohexane derivative is selected from neramexane and prodrugs, salts, isomers, analogs and derivatives thereof.

34. The method of claim 33, wherein the 1-amino-alkylcyclohexane derivative is neramexane.

35. The method of claim 30, wherein the 1-amino-alkylcyclohexane derivative is administered in an amount of 1 to 200 mg per day.

36. The method of claim 35, wherein the 1-amino-alkylcyclohexane derivative is administered in an amount of 10 to 40 mg per day.

37. A method for treating pain hypersensitivity in a living animal body, including a human, such method comprising administering to the living animal body, including a human, a therapeutically effective amount of an 1-amino-1,3,3,5,5-pentamethylcyclohexane (neramexane), or prodrug, salt, isomer, analog or derivative thereof.

38. The method of claim 37, wherein the pain hypersensitivity is hyperalgesia.

39. The method of claim 37, wherein the pain hypersensitivity is allodynia.

40. The method of claim 37, wherein the pain hypersensitivity is selected from visceral hypersensitivity, musculoskeletal allodynia/hyperalgesia and cutaneous allodynia/hyperalgesia.

41. The method of claim 40, wherein the visceral hypersensitivity is associated with disorders selected from irritable bowel syndrome (IBS), gastroesophageal reflux disease (GERD), and functional dyspepsia.

42. A method for treating neuropathic pain in a living animal body, including a human, such method comprising administering to the living animal body, including a human, a therapeutically effective amount of an 1-amino-1,3,3,5,5-pentamethylcyclohexane (neramexane) or prodrug, salt, isomer, analog, or derivative thereof.

43. The method of claim 37, wherein the 1-amino-1,3,3,5,5-pentamethylcyclohexane (neramexane) or prodrug, salt, isomer, analog, or derivative thereof is administered in an amount of 1 to 200 mg per day.

44. The method of claim 43, wherein the 1-amino-1,3,3,5,5-pentamethylcyclohexane (neramexane) or prodrug, salt, isomer, analog, or derivative thereof is administered in an amount of 10 to 40 mg per day.

45. The method of claim 37, wherein the 1-amino-1,3,3,5,5-pentamethylcyclohexane (neramexane) or prodrug, salt, isomer, analog, or derivative thereof is administered in an amount of 5 to 100 mg per day.

46. The method of claim 45, wherein the 1-amino-1,3,3,5,5-pentamethylcyclohexane (neramexane) or prodrug, salt, isomer, analog, or derivative thereof is administered in an amount of 12.5 to 80 mg per day.

47. The method of claim 42, wherein the 1-amino-1,3,3,5,5-pentamethylcyclohexane (neramexane) or prodrug, salt, isomer, analog, or derivative thereof is administered in an amount of 1 to 200 mg per day.

48. The method of claim 47, wherein the 1-amino-1,3,3,5,5-pentamethylcyclohexane (neramexane) or prodrug, salt, isomer, analog, or derivative thereof is administered in an amount of 10 to 40 mg per day.

49. The method of claim 42, wherein the 1-amino-1,3,3,5,5-pentamethylcyclohexane (neramexane) or prodrug, salt, isomer, analog, or derivative thereof is administered in an amount of 5 to 100 mg per day.

50. The method of claim 49, wherein the 1-amino-1,3,3,5,5-pentamethylcyclohexane (neramexane) or prodrug, salt, isomer, analog, or derivative thereof is administered in an amount of 12.5 to 80 mg per day.
